(19) United States
(12) Patent Application Publication
(10) Pub. No.: US 2015/0045215 A1
(43) Pub. Date: Feb. 12, 2015

(54) ARTICLES FOR IN-HOME COMPOSTING

(71) Applicant: The Procter & Gamble Company, Cincinnati, OH (US)

(72) Inventors: Jennifer Melissa Ross DEVINE, Cincinnati, OH (US); Brent William MASON, West Chester, OH (US); Jamesina Anne FITZGERALD, Trenton, OH (US); Alice Jean MICHELS, Cincinnati, OH (US)

(21) Appl. No.: 14/522,608

(22) Filed: Oct. 24, 2014

Related U.S. Application Data
(63) Continuation of application No. PCT/US2013/038092, filed on Apr. 25, 2013.
(60) Provisional application No. 61/638,509, filed on Apr. 26, 2012.
(60) Provisional application No. 61/644,541, filed on May 9, 2012.

(51) Int. Cl. A61L 9/014

(52) U.S. Cl. CPC A61L 9/014
USPC 504/101; 424/76.5

(57) ABSTRACT

Unit dose articles containing activated carbon are a convenient and economical way to control malodor resulting from in-home composting.

### FIELD OF THE INVENTION

The present invention is related to unit dose articles for use in in-home composting.

### BACKGROUND OF THE INVENTION

In-home composting appliances have been described. See e.g., US 2008/0209967 A1; JP 3601973 B2. Composting within a home provides convenience. Food scraps are simply placed in the appliance (versus discarded in the trash). The benefits of composting include the reduction of waste in landfills and an economical source of plant food. However, internal research identified that malodor resulting from the composting process may be a significant barrier to wide spread consumer adoption of in-home composting. Fitting exhaust piping to vent the appliance outside the home is expensive, complex, and usually not desired by the consumer. Use of some commercially available activated carbon air filters has only partial success in eliminating malodor. Powerful fans (and thus noisy and energy consuming) may be needed to push the exhaust through the filter. And when the lid of the composting appliance is lifted (e.g., to add food scraps etc for composting) thereby breaking the vacuum created by the exhaust fan, malodor is often emitted.

There is a need for a product that can easily be administered by the consumer that is simple to use and cost effective, and that provides effective malodor control from the composting process, and that is environmentally friendly.

Compost resulting from in-home composting is great for general purpose fertilizing. However, there is also a need to customize the compost for specific fertilizing applications. There is a further need to make customizing easy and convenient to the consumer.

### SUMMARY OF THE INVENTION

The present invention attempts to address these and any other needs. A first aspect of the invention provides an article comprising a first composition comprising from 1 g to 1,000 g of activated carbon, wherein the first composition is enclosed by a film to form the unit dose. Without wishing to be bound by theory, the activated carbon is an effective means to control malodor from the composting process. A unit dose form provides convenience to the consumer. A second aspect of the invention provides for a method of composting comprising the step of administering an article into a composting appliance, wherein the article comprises a first composition comprising from 1 g to 1,000 g of activated carbon, wherein the first composition is enclosed by a film to form the water soluble unit dose. A third aspect of the invention provides for a multi-compartment article comprising a first composition and a second composition. The first composition comprises from 1 g to 1,000 g of activated carbon, wherein the first composition is contained by a film to form a first compartment of the article. The second composition is contained by the film to form a second compartment of the article. A fourth aspect of the invention provides for a malodor treatment regimen method comprising the step of administering at least a first article comprising activated carbon and a second article comprising activated carbon into a composting appliance during a time period. The time period comprises at least a first unit of time and a second unit of time, where the first unit of time is not the same as the second unit of time. The first article is administered after a first unit of time and the second article is administered after a second unit of time. A fifth aspect of the invention provides for a method of treating malodor in a composting appliance comprising the step of administering a tablet into the composting appliance, wherein the tablet is a compressed shaped body comprising from about 1 g to about 1,000 g of activated carbon. A sixth aspect of the invention provides for a product comprising a tub and a plurality of articles, wherein the tub contains the plurality of articles. Each of the plurality articles comprises from about 1 g to about 1,000 g activated carbon. A seventh aspect of the invention provides for a product comprising a flexible sealable bag and a plurality of articles, wherein the bag contains the plurality of articles. Each of the plurality articles comprises from about 1 g to about 1000 g activated carbon. The article is selected from the group consisting of: a compressed tablet; and unit dose article comprising a biodegradable and water soluble film that contains the activated carbon. An eighth aspect of the invention provides for a starter kit comprising a composting appliance; an aforementioned article; and instructions for administering the article to the composting appliance.

A ninth aspect of the invention provides for an article comprising from about 0.1 g to about 1,000 g of a fertilizer composition wherein the fertilizer composition is contained by a biodegradable and preferably water soluble film. Without wishing to be bound by theory, adding a fertilizer composition to compost is a way to customize compost for a specific end-use. A unit dose form provides convenience to the consumer. A tenth aspect of the invention provides for a compressed tablet comprising from about 0.1 g to about 100 g of a fertilizer composition, wherein the table is a compressed shaped body. An eleventh aspect of the invention provides for a multi-compartment article comprising a first composition and a second composition. The first composition comprises a fertilizer composition that is contained by a film to form a first compartment of the article. The second composition is contained by the film to form a second compartment of the article. A twelfth aspect of the invention provides for a method of treating compost; the step of administering an aforementioned article into a composting appliance. A thirteenth aspect of the invention provides for a starter kit comprising: a composting appliance; and an aforementioned article; and instructions for administering the article to the composting appliance. A fourteenth aspect of the invention provides for a tub and a plurality of articles, wherein the tub contains the plurality of articles. Each of the plurality articles comprises from about 0.1 g to about 1,000 g of a fertilizer composition. A fifteenth aspect of the invention provides for a product comprising a flexible sealable bag and a plurality of articles, wherein the bag contains the plurality of articles. Each of the plurality articles comprises from about 1 g to about 1,000 g activated carbon. The article is selected from the group consisting of: a compressed tablet; and a unit dose article comprising a biodegradable and preferably water soluble film that contains the fertilizer composition

### DETAILED DESCRIPTION OF THE INVENTION

### Activated Carbon

The term “activated carbon” is used here in the broadest sense to include any form of carbon that can absorb odor. Without wishing to be bound by theory, it is the porosity that likely is responsible for malodor absorption. Non-limiting examples of activated carbon included activated charcoal, activated coal, and biochar.

The present invention is based, in part, on the observation that activated carbon, if added to the in-home composting appliance during the composting process, eliminates (or at least mitigates) the malodor that otherwise may result from composting. Without wishing to be bound by theory, there must be enough activated carbon to provide sufficient malodor control but not too much as the activated carbon may, for example, negatively impact the pH balance of the composting system. Another aspect of the invention is based on the observation that activated carbon, if added in a single dose, may not provide effective malodor control during the entire time period of the composting process. Without wishing to be bound by theory, the malodor binding sites in the activated carbon may become blocked from the contents of the compost/pre-compost materials. As such, one aspect of the invention is to provide unit dose containing activated carbon as a regimen (e.g., once per day for two weeks). In yet another aspect, the unit dose containing activated carbon is free or substantially free of other materials or ingredients.

The use of activated carbon also provides the added advantage of acting as a soil conditioner that can improve plant growth. Articles containing activated carbon are described. Methods of administrating activated carbon, including regimens, are provided. And lastly, kits containing various articles of the present invention are provided.

There are disadvantages in working with activated carbon. Handling activated carbon is messy. It is also inconvenient for consumers to measure the appropriate amount of activated carbon, particularly over the course of a regimen. There is a need for consumers to conveniently handle and administer activated carbon for in-home composting.

The present invention solves these problems by providing a pre-measured amount of activated carbon for in-home composting application in a convenient unit dose article. In one embodiment, the article is biodegradable. In another embodiment, the article is water soluble.

One aspect of the invention provides for unit dose article comprising 1 gram to 1,000 grams of activated carbon, wherein the activated carbon is enclosed by a biodegradable material or water soluble material to form the article.

Another aspect of the invention provides for a method for in-home composting comprising the step of administering a unit dose article containing activated carbon into an in-home composting appliance. In another embodiment, a regimen is provided wherein the unit dose comprising activated carbon is administered per a unit of time basis (e.g.,. once per day, once per two days, once per week, and the like) over the course of a time period (e.g., one week, two weeks, three weeks, one month, two months, and the like).

Yet another aspect of the invention provides for a method for in-home composting comprising the step of administering a unit dose article into an in-home composting appliance, wherein the water soluble unit dose comprises from 1 gram to 1,000 grams of activated carbon, wherein the activated carbon is at least partially enclosed, preferably entirely enclosed by a film, preferably a biodegradable film, alternatively a biodegradable water soluble film, to form the unit dose article.

Still yet another aspect of the invention provides for a method for in-home composting comprising the step of administering a unit dose article containing a composting microbe and/or a microbe supplement. In one embodiment, the activated carbon is separated or substantially separated from the microbe and/or the microbe supplement. In another embodiment, the activated carbon is combined with the microbe and/or microbe supplement in one compartment of the article. In still another embodiment, a composition is provided comprising: 30 wt % to 70 wt % of an activated carbon (alternatively 40 wt % to 60 wt %); 15 wt % to 35 wt % of composting microbes (alternatively 20 wt % to 30 wt %); and 15 wt % to 35 wt % of a microbe supplement (alternatively 20 wt % to 30 wt %).

Kits and treatment regimens are provided. In one aspect, a plurality of unit dose articles are provided as part of a treatment regimen where a first article is dosed during a first unit time; a second article is dosed during a second unit time, wherein the second unit time is subsequent to the first unit time; and wherein the regimen is over the course of a time period (e.g., one week, one month, etc). The treatment regimen may also have a third, fourth, or more articles. In one embodiment, each unit time is independently selected from about ½ to about three months, alternatively from about 1 day to about 14 days, alternatively once per day. In another embodiment, the time period is independently selected from 3 days, one week, two weeks, three weeks, one month, two months, six months, and the like. In yet another embodiment, the articles are administered on an as needed basis, i.e., when malodor is present. Kits may contain the same or different articles of the present invention.

### Composting Appliance

A composting appliance (preferably for in-home use), in the simplest sense, comprises a reacting container or designated area suitable for containing food scraps and other suitable materials for composting. The reacting container is typically from about 1 liter to about 100 liters, alternatively from 25 liters to 75 liters, alternatively from 35 liters to 65 liters, alternatively combinations thereof. The appliance may contain a lid or other similar means of enclosing/accessing the reacting container (and the contents contained therein). The lid mitigates the emission of malodor emitting from materials contained in the reacting container. The home composting appliance typically comprising a mixing means that will mix the contents of the reacting container to facilitate the composting processes (e.g., improve air flow). The mixing means may be engaged on a periodic basis or a continuous basis or a combination thereof. The appliance may optionally contain a heater means (e.g., electrical heating jacket) to heat the reacting container and/or the contents contained in the container to facilitate the composting processes since many composting microbes prefer temperatures above those of ambient (i.e., above about 21° C.). Preferably the reacting container is insulated so that heating is more efficient/economical. The lid may serve the function of preventing the contents of the container from being splattered out during the mixing process and/or keeping heat contained in the container/contents (should a heater be used). In some composting appliances, there is a curing container. In other words, some appliances have a two stage process—an initial active phase and a subsequent curing phase. The curing phase is typically longer in time than the active phase. The curing container may have the volumetric parameters as previously described for the reacting container. Non-limiting examples of composting appliances include those described in JP 3601973 B2; and US 2008/0209967 A1. In one embodiment, the articles of the present invention are administered to a composting appliance, alternatively specifically to an active container of an in-home composting appliance. In another embodiment, the articles are administered to a curing container of an in-home composting appliance. In yet other embodiments, the articles are administered to both the active container and the curing container of the in-home composting appliance. In one embodiment, in-home composting is conducted in a bin, bucket or bag, alternatively without mechanical mixing and/or without external heating.

One aspect of the invention provides for a composting appliance comprising a dispenser that is in fluid communication with the reacting container and/or curing container (or additional containers the appliance may have). The dispenser further comprises a dispensing container that is capable of containing one or more compositions of the present invention, and preferably dispensing portions of the composition to the container of the composting appliance. In one embodiment, the volume of the dispensing container is from about 10 ml to about 4,000 ml (or more), alternatively about 100 ml to about 2,000 ml, alternatively from about 500 ml to about 1,000 ml, alternatively combinations thereof. Alternatively, the dispenser is capable of containing a plurality of unit doses, alternatively a plurality of unit dose articles, of the present invention, and preferably dispensing these unit doses or unit dose articles to the container of the composting appliance. The dispensing of the composition/unit dose/article by the dispenser is actuated by one or more events. The event may be the user opening the lid or pressing a button on the appliance or a pre-determined time interval (e.g., daily) or a sensor detecting a stimulus (e.g., malodor, the weight of pre-compost being added to the reacting container, etc).

The appliance may contain more than one dispenser. There may be a first dispenser dedicated to the reacting container, and a second dispenser dedicated to the curing container. Alternatively, there may be a first dispenser dedicated to dispensing a first composition to the reacting container and a second dispenser dedicated to dispensing a second composition also to the reacting container. A third and fourth dispenser may be dedicated to dispensing a respective third and fourth composition to the curing container. Combinations of these dispensers are also contemplated.

### Compositions Comprising Activated Carbon

One aspect of the invention provides for a composition comprising activated carbon. In one embodiment, the composition comprises from about 1% to about 100% activated carbon, alternatively 80% to 99%, or 85% to 100%, or 90% to 100%, or 50% to 90%, or 50% to 99% of activated carbon, or combinations thereof. The composition may also contain other ingredients such as a plasticizer, microbes (suitable for composting), microbe supplements, enzymes, or combination thereof. In one embodiment, the composition contains about 1 g to about 1000 g, alternatively about 25 g to about 500 g, alternatively about 50 g to about 250 g, or combinations thereof of activated carbon. Without wishing to be bound by theory, this amount of activated carbon is suitable for controlling malodor in typical in-home composting appliances.

The composition may be a liquid, slurry, gel, powder, granular, or combination thereof. In one embodiment, the composition may comprise less than 10% water. Without wishing to be bound by theory, higher levels of water in the composition may have a detrimental effect on the water soluble materials used to make the articles of the present invention (in those embodiments where such materials are used).

In one embodiment, the article may comprise 2, 3, or more compartments such that different compositions may be contained with these compartments. In one embodiment, the first compartment contains activated carbon, and a second composition is contained in a second compartment (wherein preferably the second composition is free or substantially free of activated carbon). Multiple compartments may provide advantages such as separating incompatible ingredients or ease of dosing.

### Customizable Fertilizer

One aspect of the invention provides a unit dose article containing a fertilizer composition. Fertilizer compositions typically contain nitrogen (from a nitrogen source) and preferably also phosphate and potassium (from phosphate and potassium sources, respectively). Fertilizers may be designated by the content of one or more these components. The contents of these components in a fertilizer may be indicated by the N—P—K value (wherein N=nitrogen content by weight percentage, P=phosphorous content by weight percentage, and K=potassium content by weight percentage). The appropriate N—P—K value depends upon the fertilizing application, which is in turn generally dedicated by the needs of the plant and/or soil conditions. For example, a general purpose fertilizer will contain a weight ratio of 12-12-12 of N—P—K respectively, or 12 wt % of nitrogen, 12 wt % phosphorous, and 12 wt % potassium. The remaining components are typically filler.

According to certain non-limiting embodiments, the fertilizer composition of the present invention may have a N component of the N—P—K value ranging from 0 to 20, alternatively from 5-10, 10-20, 5-15, 8-20, 10-20, combination thereof.

Non-limiting examples of a nitrogen source include ammonium nitrate, ammonium sulfate, urea, ammonium phosphate, anhydrous ammonium, mixtures thereof. In one embodiment, the fertilizer composition of the present invention may contain 0-1000 grams of a nitrogen source, alternatively 0.01-100 grams, 0.1-10 grams, 0.2-0.5 grams, combinations thereof of the nitrogen source.

Non-limiting examples of a phosphorus source includes calcium phosphite or a phosphorous-containing acid (e.g., phosphorous-containing acids include phosphorous acid, hypophosphorous acid, polyphosphorous acid, polyhypophosphorous acid, or mixtures thereof), or mixtures thereof. In one embodiment, the fertilizer composition of the present invention may contain 0-1000 grams of a phosphorus source, alternatively 0.01-100 grams, 0.1-10 grams, 0.2-0.5 grams, or combinations thereof of the phosphorus source.

Non-limiting examples of a potassium source include potassium chloride, potash, or mixtures thereof. In one embodiment, the fertilizer composition of the present invention may contain 0-1000 grams of a potassium source, alternatively 0.01-100 grams, 0.1-10 grams, 0.2-0.5 grams, or combinations thereof of the potassium source.

The fertilizer composition of the present invention may also contain additional nutrients such as calcium, magnesium, and sulfur from respective sources. The fertilizer compositions of the present invention may also contain micronutrients such as boron, copper, and iron—also from respective sources.

Sources of NPK may be organic, or synthetic, or combinations thereof. In one embodiment, the source of NPK is synthetic, alternatively predominantly synthetic. Without wishing to be bound by theory, synthetic sources of NPK are generally more readily available to plants where organic sources are not. In this embodiment, an ideal balance between organic and synthetic sources of fertilizer may be provided. The compost resulting from in-home composting will act as an organic source of fertilizer. Supplementing the in-home compost with the unit doses of synthetic sources of fertilizer will provide the balance of readily available and slow release sources of nutrients to plants.

Another advantage of the present invention is the many commercially available fertilizers, particularly synthetic sources, will contain filler. Use of filler increases manufacturing and transportation costs. In one embodiment, the water soluble unit doses of the present invention are substantially free of fillers. Filler is generally an inert material such as sand, lime, ground corn cobs, etc.

In one embodiment, the fertilizer composition of the present invention comprises a lysine source. Non-limiting examples of a lysine source include lysine monohydrochloride, lyside hydrate, lysine dihydrochloride, lysidine sulfate, or combinations thereof. Lysine sources may be obtained commercially from Archer-Daniels-Midland Company, Decatur, Ill.

In one embodiment, the composition contains an alkalinity source or an acidifying source. An alkalinity source includes lime or wood ash. Acidifying source includes sulfur or aluminum sulfate.

### Enzymes

One aspect of the invention provides for compositions that comprise one or more enzymes to assist in the composting process. The enzymes may be used alone or in combination with composting microbes. Examples of enzymes include: cellulases, hemicellulases, peroxidases, proteases, gluco-amylases, amylases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase or mixtures thereof. In one embodiment, the enzymes include protease, amylase, lipase, peroxidases, cutinase and/or cellulase in conjunction with one or more plant cell wall degrading enzymes. In another embodiment, the enzymes are at levels from 0.0001% to 2% by weight of the composition. A supplier of enzymes may include Star Engineering Co. Ltd., Japan.

### Unit Dose Articles

Another aspect of the invention provides for containing the aforementioned compositions in desired quantities in a unit dose article. The “unit dose article” may be a packet, a tablet, or a tablet within a packet. The unit dose article comprises one or more of the compositions of the present invention.

### Packet

The term “packet” is used in the broadest sense to include a pouch, capsule, bag, sachet, or the like. The packet comprises a compartment which contains a composition, tablet, or combination therein. The contents contained in the compartment are typically separated from the outside environment by a barrier of material. A variety of shapes and sizes of the packet are contemplated. The material that is used to form a compartment of the article may be in the form of a film. The term “film” is used broadly to include a shell, sheet, or the like. The film may be formed by casting, blow-molding, extrusion, or blow extrusion of the material. The material may be non-biodegradable, biodegradable, water soluble, or combinations thereof. In one embodiment, the material is a polymeric material, preferably water soluble and biodegradable.

Non-limiting examples of polymers, copolymers or derivatives thereof suitable for use as material for encapsulating compositions to form the unit dose articles of the present invention are selected from polyvinyl alcohols, partially hydrolyzed polyvinylacetates, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, natural gums such as xanthum and carragum, and combinations therein. In one embodiment, polymers are selected from polyacrylates and water-soluble acrylate copolymers, methylcellulose, carboxymethylcellulose sodium, dextrin, ethylcellulose, hydroxyethyl cellulose, hydroxypropyl methylcellulose, hydroxybutyl methylcellulose, maltodextrin, and polymethacrylates. In another embodiment, the polymers are selected from polyvinyl alcohols, polyvinyl alcohol copolymers, partially hydrolysed polyvinylacetates and hydroxypropyl methyl cellulose (HPMC), hydroxybutyl methylcellulose (HBMC), and combinations thereof. The polymer can have any weight average molecular weight. In one embodiment, the weight average molecular weight is from about 1,000 to about 1,000,000, alternatively from about 10,000 to about 300,000, alternatively from about 20,000 to about 150,000. Polyvinyl alcohol films are known under the trade reference Monosol and sold by Chris-Craft Industrial Products of Gary, Ind., U.S.A.

Other non-limiting examples of material that may be suitable for containing the compositions of the present invention include: gelatin, starch, polyvinyl alcohol, polyethylene glycol, cellulose ethers, gelatin, alginic acid or pectinic acid, or the like, or combinations thereof. The material may optionally contain additives adjusting its elasticity and water solubility. In another embodiment the material comprises a low melting biodegradable polyester (e.g., polycaprolactone)—either as a film or as an extrusion coated on to paper. In yet another embodiment the material comprises a polybutylene succinate or a copolymer containing the same —either as a film or an extrusion coated on to paper.

The unit dose article may have a single compartment or multiple compartments. With a multiple compartment embodiment, the unit dose article may contain different compositions in each compartment. Incompatible compositions may be separated in different compartments. In one embodiment, the compartments of the multi-compartment article may be superposed to one another.

The compositions of the present invention may be enclosed by materials by conventional methods, e.g., by using conventional apparatus with rotating drums. See e.g., U.S. Pat. Nos. 6,898,921; 5,617,710; 5,317,849; 5,188,688; 4,940,499; 4,581,875; 4,478,658; and 4,089,152. Thermoforming and injection molding are other conventional methods.

In one embodiment, the article is in the form of a square or rectangle packet. The material is a water soluble biodegradable material and is in the form of a sheet. A single sheet is folded or two sheets are sealed together at the edge region either by means of adhesive or heat-sealing. In one example, a rectangle is formed from a single folded sheet sealed on three sides, with the fourth side sealed after filling the unit dose with activated carbon. Without wishing to be bound by theory, a rectangular-shaped packet is more easily manufactured and sealed than other configurations when using conventional packaging equipment. In another embodiment, the material is a polyvinyl alcohol containing film available from MonoSol, LLC, Merrillville, Ind. In yet another embodiment, the thickness of the polyvinyl alcohol containing film is from about 10 μm to about 1,000 μm, alternatively from 20 μm to about 500 μm, alternatively combination thereof. In yet still another embodiment, the volume contained in a compartment is from 0.1 cm³ to 100 cm³, alternatively from 1 cm³ to 5 cm³, alternatively combinations thereof. A process for making thermo-formed articles is described in WO 00/55045. The film can be made by injection molding as described in WO 02/092456. A pouch making unit, for example, can be a rotator drum, as described in U.S. Pat. No. 3,057,127.

In one aspect of the invention, the article is a multi-compartment article comprising two, three, four, or more compartments. Each compartment may contain different or the same compositions. Without wishing to be bound by theory, multiple compartments allow incompatible compositions to be contained together in one convenient unit dose. Multiple compartment unit dose articles are described, for example, in U.S. Pat. No. 7,125,828. Methods of making multiple compartment unit dose articles are described, for example, in US 2002/0169092 and US 2009/0199877 A1.

In one embodiment, the composition, contained in the water soluble material, comprises a plasticizer. In one embodiment, the plasticizer is a polyhydric alcohol (such as alkylene glycols (e.g., ethylene glycol, propylene glycol)), glycerol, polyols, sorbitol, or combinations thereof. In another embodiment, the composition comprises from about 1% to about 50%, alternatively from about 8% to about 12%, or combinations thereof, of plasticizer by weight of the composition. Without wishing to be bound by theory, plasticizers may improve the flexibility and resistance to cracking of the water soluble materials used herein.

### Tablet

One aspect of the invention provides for forming the compositions of the present invention into a tablet. The tablet may be administered to the composter. Alternatively the tablet may be contained in a compartment of a packet, wherein the packet may be administered to the composter. One advantage of the packet is that the user does not touch or come into direct contact with the tablet (or other compositions of the present invention).

From the manufacturing viewpoint, multi-compartment pouches containing one or more tablets are very convenient because the filling of pouches with a particulate composition can be complex and prone to inaccuracies. It is often slow and likely to produce dust, such that it can be very difficult to avoid dust deposition on the pouch seal area. This can be detrimental to achieving a strong seal.

The tablet can be formed using any suitable method, but preferably by compression, for example in a tablet press. Preferably, the tablet is a compressed shaped body prepared by mixing together the components of the present invention followed by applying a compression pressure of at least about 10 kg/cm², alternatively at least about 20 kg/cm², or 40 kg/cm², or 80 kg/cm², or 125 kg/cm², or about 250 kg/cm², or at least about 350 kg/cm² (3.43 kN/cm²). In one embodiment, the compression pressure is from about 400 kg/cm² to about 2,000 kg/cm², alternatively from about 600 kg/cm² to about 1200 kg/cm² , or combinations thereof. “Compression pressure” herein is the applied force divided by the cross-sectional area of the tablet in a plane transverse to the applied force—in effect, the transverse cross-sectional area of the die of the rotary press. In another embodiment, the tablet has a Child Bite Strength (“CBS”) test of at least about 6 kg, alternatively greater than about 8 kg, or about 10 kg, or about 12 kg, and/or greater than about 14 kg, or combinations thereof. In one embodiment, the tablet has a CBS test of less than 1,000 kg, alternatively less than about 500 kg. CBS being measured per the US Consumer Product Safety Commission Test Specification.

The tablet can take a variety of geometric shapes such as spheres, cubes, etc but preferably has a generally axially-symmetric form with a generally round, square or rectangular cross-section.

In one aspect of the invention, the tablet is prepared such that it comprises at least one mould in its surface. The mould can contain a non-compressed portion. The non-compressed portion of the tablet may be in solid, gel, or liquid form.

The mould or moulds can also vary in size and shape and in their location, orientation and topology relative to the tablet. For example, the mould or moulds can be generally circular, square or oval in cross-section; they can form an internally-closed cavity, depression or recess in the surface of the tablet, or they can extend between unconnected regions of the tablet surface (for example axially-opposed facing surfaces) to form one or more topological ‘holes’ in the tablet; and they can be axially or otherwise symmetrically-disposed relative to the tablet or they can be asymmetrically disposed. Preferably, the mould is preformed, for example being created using a specially designed tablet press wherein the surface of the punch that contacts the detergent composition is shaped such that when it contacts and presses the composition it presses a mould, or multiple moulds into the tablet. Preferably, the mould will have an inwardly concave or generally concave surface to provide improved housing and physical storage of the non-compressed portion of the tablet. Alternatively, the mould can be created by compressing a preformed body of composition disposed annularly around a central dye, thereby forming a shaped body having a mould in the form of a cavity extending axially between opposing surfaces of the body. Tablets with moulds are very useful from the viewpoint of accommodating the compartment comprising the liquid, gel or paste compositions of the present invention.

In one embodiment, the compressed portion, non-compressed portion, or combination thereof, may contain activated carbon, fertilizer, composting microbe, microbe supplement, perfume, or combination thereof.

Methods of making tablets comprising a compressed portion and a non-compressed portion are described, for example, in U.S. Pat. No. 6,399,564 B1 (e.g., col. 35,1. 59-col. 37,1. 5). In one embodiment, the non-compressed portion contains a carrier component. A preferred carrier component is an organic polymer, preferably solid at ambient temperatures. An example of a carrier component is polyethylene glycol (PEG). The non-compressed portion may contain other ingredients such as solvents, binding agents, gelling agents, and/or thickening agents, See U.S. Pat. No. 6,399,564 at col. 5,1. 26 to col. 9, 1. 3. The non-compressed portion may be coated with a coating layer. The coating may be used to affix the non-compressed portion to the compressed portion. See U.S. Pat. No. 6,399,564 at col. 9,1. 4-35.

The compressed portion of the tablet may comprise a disrupting agent. The disrupting agent may be a disintegrating or effervescing agent. See U.S. Pat. No. 6,399,564 at col. 4, 1. 1-18.

A tablet of the present invention may comprise a weight from about 1 g to about 1,000 g, alternatively from about 100 g to about 2,000 g, alternatively from about 250 g to about 1,500 g, alternatively from about 500 g to about 1,000 g, alternatively combinations thereof.

A tablet of the present invention may comprise a volume from 0.1 cm³ to about 1,000 cm³, alternatively from about 100 cm³ to about 2,000 cm³, alternatively from about 250 cm³ to about 1,5000 cm³, alternatively from about 500 cm³ to about 1,000 cm³, alternatively combinations thereof.

### Kits

One aspect of the invention provides for a kit, wherein the kit comprises at least one unit dose article of the present invention and optionally instructions for use. In one embodiment, the kit comprises a plurality of unit dose articles wherein a first unit dose comprises a first fertilizer composition and a second unit dose comprises a second fertilizer composition, wherein the first fertilizer composition is different from the second fertilizer composition. These compositions are different based on different applications. For example, a first unit dose article may be directed to the spring when annual plants generally need more phosphorous for root growth, whereas a second unit dose article may be directed for use in the summer when plants generally need more nitrogen for leaf growth. In another example, the first unit dose article may be directed to an acid loving plant whereas a second unit dose article is directed to a non-acid loving plant. In yet still another example, a first unit dose article may be directed to a vegetable bearing plant and a second unit dose article be directed to a fruit bearing plant.

One aspect of the invention provides for a kit comprising at least one unit dose article containing activated carbon and at least one unit dose article containing a fertilizer composition. In this aspect of the invention, the consumer is provided two types of articles; a first type to facilitate the composting process by providing malodor control (and optionally composting microbes); and a second type to customize the compost for a specific fertilizer application supplementing the compost providing a desired N—P—K value. In one embodiment, the article contains both the activated carbon and a fertilizer composition.

Alternatively a product array is provided wherein a first product contains a unit dose article containing activated carbon and a second product comprises a unit dose article containing a fertilizer composition.

In one embodiment, a kit comprising a plurality of articles is provided. The articles may be the same or different. An article may contain only activated carbon or an article may contain only a fertilizer composition. An article may contain both activated carbon and a fertilizer composition. In one embodiment, the articles of the kit are the same, and wherein each article of the plurality of articles each contains activated carbon and a fertilizer composition. In another embodiment, the kit comprises instructions instructing the user to administer the article to a composting appliance according to a regimen. In yet another embodiment the treatment regimen comprises a time period wherein at least two units of time are different for the time period (i.e., article administration frequency changes). For example, the regimen may have a time period of about two months. The first, second, third, and fourth units of time may be once per week, where as the final fifth unit of time is about one month (to complete the time period of about two months).

### Composting Microbes and Microbe Supplements

In one aspect of the present invention, the composition may comprise a composting microbe (e.g., bacteria, yeast, mold fungi/spores/hyphal suspensions, or combinations thereof) and optionally a microbe supplement.

The term “microbe supplement” includes microbes, probiotics, antibiotic, biocide, or combinations thereof. A prebiotic is a chemical that selectively encourages the growth of desired microbes and/or discourages the growth of undesired microbes. Non-limiting examples of a prebiotic include: selective food carbons (sugars such as fructooligosaccharides, certain alcohols (e.g. inositol, mannitol), certain selective essential cations (such as iron, vanadium), or cofactors such as hemin or Vitamin K). A probiotic is a desirable microbe that outcompetes and/or selectively inhibits undesirable microbes. A non-limiting example of a prebiotic is a lactic acid bacteria producing lactic acid that inhibit gram negative pathogens. Antibiotics/Biocides are natural or chemical agents that act directly on undesirable microbes to inhibit their growth or kill them.

The composition containing a composting microbe may be contained in a unit dose article. Microbes can be added to aid in one or more of the following functions: accelerate the onset of aerobic decomposition of food and vegetative wastes (contained in the pre-compost); provide malodor control; potentiate high decomposition activity in terms of rate and spectrum of decomposition on a wide range of organic materials in the pre-compost and compost, including high cellulose and fibrous plant/paper materials; improve composting hygiene; reinforce the establishment/colonization of bacteria to fuel high intensity aerobic decomposition; raise the temperature of the organic compost material to enable thermophilic decomposition; aid in reducing or eliminating the undesired or pathogenic microbes; increase the quality of the final compost (e.g., pH, percentage of humus, moisture retention, bioavailability of micronutrients, and nutrient content); or combinations thereof.

Composting microbes can be administered into the composting appliance by the articles of the present invention a number of ways. The user can simply add the composting microbes and/or microbe supplements before, during, and after pre-compost is added to the composting appliance. Alternatively, the composting microbes/supplements are added before initiating the operation of the machine. In one embodiment, the composting microbes or microbe supplements are added to the composting appliance according to a treatment regimen (as previously described).

Microbes/supplements can be administered as a solution or liquid suspension, or dry form (e.g., powder). In addition to water, the suspension/solution may contain other ingredients including solvents, dispersing agents, stabilizers, toxicity modifiers, pH buffers etc. Generally, about 1 billion microbial cells are contained: per one milliliter of solution/suspension; or one milligram in a dry form. The solution/suspension can be administered via a water soluble unit dose article of the present invention.

A listing of microbes may include the following: bacteria of the Bacillus genus; Bacillus smithii (preferably the Oklin strain); Bacillus sulfolobus (preferably the Oklin strain); Bacillus subtilis; Bacillus licheniformis; Bacillus megaterium; Bacillus polymyxa; Bacillus circulans; Bascillus cereus; bacteria of Lactobacillus genus; Lactobacillus acidophilus, Lactobacillus actinomyces; Lactobascillus casei; Lactobacillus Streptomyces; Lactobacillus frankia; Lactobacillus Azotobacter; and combinations thereof.

Microbes may contain fungi, preferably in combination with bacteria. A listing of microbes may include the following: fungi of the Aspergillus genus; Aspergillus niger; Aspergillus orizae; fungi of the Rhizopus genus, fungi of the Saccaromyces genus; fungi of the Trichondema genus; and combinations thereof.

Examples of supplements may include ingredients to facilitate composting and/or provide better compost. Ingredients that may help facilitate composting include high nitrogen containing components such as soybean and alfalfa (to encourage microbial growth); sawdust (increase circulation therefore oxygen content); trace minerals; sugars (e.g., molasses to help fuel microbes); enzymes (e.g., proteases, lipases, cellulase, lignase, pectinase, etc.), and mixtures thereof. Ingredients that may help in providing better compost include soil conditions (e.g., humus and humic leonardite compounds derived from leonardite), rooting hormones, or combinations thereof.

One aspect of the invention provides for a method of administering a composition to the in-home composting appliance, wherein the composition comprises a microbe and/or microbe supplement.

The articles and/or compositions of the present invention may contain a perfume. Perfumes are disclosed in the US patent publication ion co-filed patent application P&G Case 12404P.

### Secondary Packaging

The unit dose article(s) of the present invention may be contained in a secondary package. In one embodiment, a container system is provided comprising: a tub comprising rigid or semi-rigid walls and a lid, wherein the lid is capable of opening to provide access to an interior volume of the tub. Preferably the lid is capable of closing after being opened. The interior volume contains at least one, preferably a plurality of the unit dose articles. The tub and lid may be made from plastic. In one embodiment, the tub comprises a front wall, a back wall (opposite the front wall), side walls (extending between the front wall and the back wall); a bottom (extending between the front wall, back wall, and side walls). In another embodiment, the lid in its closed position, extends between the front wall, back wall, and side wall(s) and is opposite the bottom (i.e., forms the top). The lid may comprise a hinge. See e.g., US 2011/0204087 A1.

In another embodiment, a container system comprises a flexible sealable bag containing at least one, preferably a plurality, of the unit dose articles of the present invention. The bag comprises a front panel and a back panel marginally joined thereto along at least two opposed edges in a confronting relationship to form a closed bag, and the front panel and the back panel being joined at the top by a frangible seal. A bottom panel is between and joins the front panel and the back panel to form an interior volume (to contain the article(s)). In yet another embodiment, the front panel of the bag comprises at least one transparent window portion where one or more of the unit dose articles contained inside the bag are viewable from the outside of the bag. See e.g., U.S. patent publication from U.S. application Ser. No. 12/885,793, filed Sept. 20, 2010 (P&G Case 11880).

In one embodiment, the interior volume of the container of the container system (empty from any contents) comprises a volume from about 50 cm³ to about 4,000 cm³, alternatively from about 500 cm³ to about 2,000 cm³, alternatively from about 1,000 cm³ to about 3,000 cm³, alternatively combinations thereof. In another embodiment, the container of the container system contains from about 1 to about 150 unit dose articles, alternatively from about 6 to about 100, alternatively from about 12 to about 50, alternatively from about 15 to about 25, alternatively combinations thereof. In yet another embodiment, the container of the container system comprises a foot print area from about 25 cm² to about 500 cm², alternatively from about 50 cm² to about 400 cm², alternatively from about 75 cm² to about 300 cm², alternatively from about 100 cm² to about 200 cm², alternatively from about 150 cm² to about 250 cm², alternatively combinations thereof.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multi-compartment article comprising a first composition and a second composition,
wherein the first composition comprises from 1 g to 1,000 g of activated carbon, and
wherein the first composition is contained by a film to form a first compartment of the article; and
wherein the second composition is contained by the film to form a second compartment of the article.

2. The article of claim 1, wherein the second composition is substantially free of the activated carbon.

3. The article of claim 1, wherein the second composition comprises microbes suitable for composting, or a microbe micronutrient, or mixture thereof.

4. A starter kit comprising:
(a) a composting appliance; and
(b) an article according to claim 1; and
(c) optionally instructions for administering the article to the composting appliance.

5. A malodor treatment regimen method comprising the step of administering at least a first article comprising activated carbon and a second article comprising activated carbon into a composting appliance during the time period,
wherein the time period comprises at least a first unit of time and a second unit of time,
wherein the first unit of time is not the same as the second unit of time;
wherein the first article is administered after a first unit of time and the second article is administered after a second unit of time.

6. The method of claim 5, wherein the method further comprises the step of administering a third article comprising activated carbon to the composting appliance during the time period and after the second article is administered.

7. The method of claim 6, wherein the method further comprises the first unit of time and second unit of time independently selected from the group consisting of from about 1 day, 2 days, 3 days, 4 days, 5 days, 6 days, one week, two weeks, or combinations thereof; and wherein the time period is selected from the group consisting of from about two weeks, three weeks, four weeks, five weeks, 6 weeks, 7 weeks, 8 weeks, or combinations thereof.

8. A method of treating malodor in a composting appliance comprising the step of administering a tablet into the composting appliance, wherein the tablet is a compressed shaped body comprising from about 1 g to about 1,000 g of activated carbon.

9. The method of claim 8, wherein the tablet is made by applying a compression pressure of at least 10 kg/cm2.

10. The method of claim 8, wherein the tablet comprises about an axially-symmetric form.

11. The method of claim 10, wherein the tablet further comprises a non-compressed portion.

12. The method of claim 11, wherein the non-compressed portion further contains an ingredient selected from the consisting of: composting microbe, microbe supplement, and a combination thereof.

13. The method of claim 10, wherein the tablet further comprises a fertilizer.

14. The method of claim 10, wherein the tablet further comprises a component selected from the consisting of: composting microbe, microbe supplement, and a combination thereof.
